(19) United States
(12) Patent Application Publication
(10) Pub. No.: US 2018/0085000 A1
(43) Pub. Date: Mar. 29, 2018

(54) SYSTEM AND METHOD FOR AUTOMATIC PREDICTION AND PREVENTION OF MIGRAINE AND/OR EPILEPSY

(71) Applicant: KONINKLIJKE PHILIPS N.V., ElNDHOVEN (NL)

(72) Inventors: MIRELA ALINA WEFFERS-ALBU, BOUKOUL (NL); RAYMOND VAN EE, GELDROP (NL)

(21) Appl. No.: 15/562,569

(22) PCT Filed: Mar. 24, 2016
(86) PCT No.: PCT/EP2016/056473

§ 371 Date: Sep. 28, 2017

(30) Foreign Application Priority Data
Mar. 31, 2015 (EP) 15161858.4

(51) Int. Cl. A61B 5/00; A61B 5/0476

(52) U.S. Cl. CPC A61B 5/0042; A61B 5/0006; A61B 2562/029; A61B 2562/0271; A61B 2562/0247; A61B 5/0476

(57) ABSTRACT

The present invention relates to the field of detecting/preventing migraine attacks. In particular, a system and method for detecting and preventing a migraine episode in a user are provided. The system comprises a brain activity sensor for detecting a brain activity signal; a migraine trigger detection unit for detecting potential migraine triggers, said potential migraine triggers being circumstances that may potentially cause a migraine attack in the user. A processing unit is configured to: process the brain activity signal to identify one or more signal characteristics in the brain activity signal that are indicative of an upcoming migraine attack in the user; identify one or more correlations between the one or more identified signal characteristics in the brain activity signal and the detected potential migraine triggers; determine, based on the identified one or more correlations, one or more personal migraine triggers that are likely to cause a migraine attack in the user; an output unit for providing the user with a personalized feedback which informs the user about the determined one or more personal migraine triggers and/or how the user may prevent the determined one or more personal migraine triggers.

### FIELD OF THE INVENTION

The present invention relates to detecting and preventing migraine episodes and/or epilepsy seizure episodes in a user. A system and method are provided wherein brain activity of the user and potential migraine triggers and/or potential epilepsy triggers are detected and analysed. A personal feedback informs the user about the personal migraine triggers and/or personal epilepsy triggers. In addition or alternatively, the personal feedback may inform how the user may prevent the personal migraine triggers and/or personal epilepsy triggers.

### BACKGROUND OF THE INVENTION

Both migraine and epilepsy are heterogeneous families of chronic disorders with highly variable clinical features, natural histories, and patterns of treatment response. Both are characterized by episodes of neurologic dysfunction, sometimes accompanied by headache, as well as gastrointestinal, autonomic, and psychological features. Each has an internationally recognized classification system.

Migraine is a chronic neurological disease characterized by recurrent headaches of different severity and which is often in association with a number of autonomic nervous system symptoms. Associated symptoms may include nausea, vomiting, sensitivity to light, sound or smell. The pain is generally worsened by performing physical activity. It is assumed that migraines arise due to a mixture of environmental and genetic factors.

Treatment of migraine usually requires intake of medicaments, such as Topiramate. These medicaments may involve serious side-effects. Alternative therapies, such as acupuncture, physiotherapy and massage, are used. Migraine surgery is also employed in severe cases involving the compression of certain nerves.

According to the World Health Organization “headaches and migraines are under-recognized and under-diagnosed and it is costing the world's economy £140 billion a year. Almost half of all adults worldwide suffer headache disorders and a burden being imposed on society must be improved, the WHO said. Publishing its first global atlas on headaches, the Geneva-based United Nations health body said it found that 47% of adults have a headache disorder and “the financial costs to society through lost productivity are enormous” (http://www.dailymail.co.uk/health/article-1383437/Headaches-migraines-cost-economy-140 billion-says-World-Health-Organisation.html). In the European Union alone, 190 million days are lost from work every year because migraine, it said.”

In the context of elderly people, migraine treatment presents special challenges due to the fact that the elderly population typically suffers from additional co-morbid diseases which may prohibit the use of some medications. In that sense older migraine patients require particular care in treatments to take into account potential pharmacological interactions with other drugs that are necessary to manage their ongoing chronic conditions.

Epilepsy affects many people and is accompagnied by the occurrence of spontaneous seizures. Anticonvulsant medications can be administered to many patients for preventing seizures, but patients often suffer side effects. For 20-40% of patients with epilepsy, medications are not effective—and even after surgical removal of epilepsy-causing brain tissue, many patients continue to experience spontaneous seizures. Despite the fact that seizures occur infrequently, patients with epilepsy experience persistent anxiety due to the possibility of a seizure occurring. Clinicians who care for patients with epilepsy have long known that many patients are aware of periods when seizures are more likely, though they can rarely specify an exact time when seizures will happen.

Epilepsy seizures bring in addition to the discomfort and hazard incurred during their progression, further negative after effects that impact significantly patient health and quality of life. A first well-known example is that epileptic seizures can make patients more prone to falls and injuries (http://www.healthline.com/health/epilepsy/effects-on-body). In the general patient population, according to the Epilepsy Foundation Michigan, about 30% of people with epilepsy eventually develop clinical depression.

Epilepsy in elderly chronic patients poses several additional problems (The Role of Primary Care in Epilepsy Management, Epilepsy Action, 2005; http://www.patient.co.uk/doctor/Epilepsy-in-Elderly-People.htm) including diagnostic difficulties; susceptibility to anti-epileptic drug (AED) side-effects and toxicity, and increased likelihood of interaction with other medication necessary for treating other existing chronic conditions; social difficulties and physical restrictions to lifestyle—leading to increased isolation; increased impact of driving restrictions—leading to dependency on others for being brought to consultations and therapy sessions which implies that patients are often less adherent to their treatment plan; seizures that cause falls are more likely to cause serious injury in older people that can in aftermath—given the already fragile health of these patients—lead to death; and multidisciplinary service requirements in the community, including liaison nurse, social worker and occupational therapist. Given the significant impact on elderly chronic patients health and quality of life as well as the complications related to seizure treatment of elderly chronical patients it becomes very relevant to bring forward solutions that are able to predict the onset of seizures and support patients to prevent them.

US 2003/0144829 A1 pertains to a system having a body-worn sensor unit predicting the onset of a chronic symptom, such as migraine, in a person and alerting the person about the possible onset of the chronic symptom. In response, an appropriate drug or other therapy may be automatically administered. Various types of sensors may be used in the body-worn sensor unit, such as sensors for measuring physiological parameters including temperature, physical activity, and patient specific parameters possibly affecting the patient, such as pressure, temperature and light levels. A modeling agent is employed which makes a predictive determination as to the onset of symptoms. The modeling agent may train a neural network model.

US 2013/0066395 A1 discloses a device and method for producing prophylactic or therapeutic effects in a patient by utilizing an energy source that transmits energy non-invasively to nervous tissue. An imminent medical attack, such as a migraine attack, may be forecasted by acquiring a training set of ambulatory recordings using for instance EEG sensors for mapping the prefrontal cortex activation and other ambulatory sensors indicative of possible triggering events, such as environmental, physiological, and cognitive sets of events. All recordings are used to parameterize a model that predicts and warns the patient about the imminent onset of the migraine attack. A support vector machine may be trained and used to sound an alarm and advise the use of vagus nerve stimulation, whenever there is the forecast of an imminent attack.

The current solutions for detection and treatment of migraine and epilepsy have the drawback that they essentially aim on predicting the onset of the ailment in order to provide early treatment for possibly avoiding strong symptoms and/or the onset of ailment. Solutions according to the state of the art are often reactive, in the sense that they come mainly in the form of treatments or cures that aim to elevate the migraine or epilepsy once onset is established. The known pharmacological treatments are potentially hazardous to the patient since side-effects and toxicity from medication interaction with other drugs used to treat different chronic conditions, such as in older patients, may occur. The current systems do neither identify migraine triggers to support patients to avoid these triggers in future nor are they able to really prevent migraine onset. Likewise, the current systems do neither identify epilepsy triggers to support patients to avoid these triggers in future nor are they able to really prevent epilepsy onset. Interventions which may tackle the triggers for migraine or epilepsy are not provided by the current systems. The current systems furthermore do not provide personailzed solutions for tackling migraine or epilepsy.

### SUMMARY OF THE INVENTION

It is an object of the present invention to provide a system and method for predicting migraine or epilepsy onset in a user and helping the user to prevent the onset. Another object of the present invention resides in the provision of a system and method which does not introduce risks associated with medication interaction and which is not hazardous. Still another objective of the present invention is to provide a personalized solution which takes care about the personal circumstances of each patient in terms of age and pre-existing illnesses. Still a further objective is the provision of a system and method effectively giving a patient the possibility to identify and avoid personal migraine triggers and/or personal epilepsy triggers. Another objective resides in the provision of a system and method effectively giving a patient the possibility to avoid migraine episodes and/or epilepsy seizure episodes.

In a first aspect of the present invention a system for detecting and preventing a migraine episode in a user is provided.

The system comprises:

a brain activity sensor for detecting a brain activity signal;

a migraine trigger detection unit for detecting potential migraine triggers, said potential migraine triggers being circumstances that may potentially cause a migraine attack in the user;

a processing unit configured to:

(i) process the brain activity signal to identify one or more signal characteristics in the brain activity signal that are indicative of an upcoming migraine attack in the user;

(ii) identify one or more correlations between the one or more identified signal characteristics in the brain activity signal and the detected potential migraine triggers;

(iii) determine, based on the identified one or more correlations, one or more personal migraine triggers that indeed or with a certain probability cause a migraine attack in the user;

an output unit for providing the user with a personalized feedback informing the user about the determined one or more personal migraine triggers and/or how the user may prevent the determined one or more personal migraine triggers.

In a further aspect of the present invention a system for detecting and preventing an epilepsy seizure episode in a user is provided.

The system comprises:

a brain activity sensor for detecting a brain activity signal;

an epilepsy trigger detection unit for detecting potential epilepsy triggers, said potential epilepsy triggers being circumstances that may potentially cause an epileptic attack in the user;

a processing unit configured to:

(i) process the brain activity signal to identify one or more signal characteristics in the brain activity signal that are indicative of an upcoming epileptic attack in the user;

(ii) identify one or more correlations between the one or more identified signal characteristics in the brain activity signal and the detected potential epilepsy triggers;

(iii) determine, based on the identified one or more correlations, one or more personal epilepsy triggers that indeed or with a certain probability cause an epileptic attack in the user;

an output unit for providing the user with a personalized feedback informing the user about the determined one or more personal epilepsy triggers and/or how the user may prevent the determined one or more epilepsy triggers.

In still a further aspect of the present invention a method for detecting and preventing a migraine episode in a user is provided. Said method comprises the steps of:

detecting a brain activity signal of a user;

processing the brain activity signal to identify one or more signal characteristics in the brain activity signal that are indicative of an upcoming migraine attack in the user;

detecting potential migraine triggers, said potential migraine triggers being circumstances that may potentially cause a migraine attack in the user;

identifying one or more correlations between the one or more identified signal characteristics in the brain activity signal and the detected potential migraine triggers;

determining, based on the identified one or more correlations, one or more personal migraine triggers that indeed or with a certain probability cause a migraine attack in the user;

providing the user with a personalized feedback informing the user about the determined one or more personal migraine triggers and/or how the user may prevent the migraine onset by countering the determined one or more personal migraine triggers.

In yet another aspect of the present invention a method for detecting and preventing an epilepsy seizure episode in a user is provided. Said method comprises the steps of:

detecting a brain activity signal of a user;

processing the brain activity signal to identify one or more signal characteristics in the brain activity signal that are indicative of an upcoming epileptic attack in the user;

detecting potential epilepsy triggers, said potential epilepsy triggers being circumstances that may potentially cause an epileptic attack in the user;

identifying one or more correlations between the one or more identified signal characteristics in the brain activity signal and the detected potential epilepsy triggers;

determining, based on the identified one or more correlations, one or more personal epilepsy triggers that indeed or with a certain probability cause an epileptic attack in the user;

providing the user with a personalized feedback informing the user about the determined one or more personal epilepsy triggers and/or how the user may prevent the epilepsy onset by countering the determined one or more personal epilepsy triggers.

The herein presented system and method tests the brain activity of the user in order to determine the likelihood and time frame of a migraine and/or epilepsy seizure onset. This is performed by the brain activity sensor which detects and optionally records a brain activity signal, such as an electroencephalography (EEG) signal, functional near-infrared spectroscopy (fNIRS) or functional magnetic resonance imaging (fMRI). The brain activity signal encompasses characteristics reflecting the onset of a migraine episode in closer future. Such characteristics are particular wave(s), wave form(s), peak(s), peak area(s) and peak envelope(s) within the brain activity signal indicating the development of migraine and/or epilepsy seizure in closer future.

The one or more signal characteristics in the brain activity signal that are indicative of an upcoming migraine attack and/or epileptic attack require detecting the brain activity signal for a rather short period of time and low repetition frequency.

In addition, the present system and method identifies potential migraine triggers including all circumstances that may potentially cause a migraine attack in the user which may be found in the personal living conditions. These potential migraine triggers are monitored along the user's day to possibly cover each conceivable, yet unknown factual migraine trigger. Each of the detected potential migraine triggers is subjected, alone or in combination with one or more other potential migraine triggers, to a correlation with the characteristics in the brain activity signal. By correlation of the one or more identified signal characteristics in the brain activity signal and the detected potential migraine triggers, the system and method reveals the one or more personal migraine triggers, i.e. migraine triggers that do actually cause migraine in the user at hand.

Repeated correlation of signal characteristics obtained from additional measurements with already identified personal migraine triggers, further potential migraine triggers and potential migraine triggers which are suspected to be factual personal migraine triggers (i.e. unconfident personal migraine triggers wherein still further testing is required) permits teaching the system and method. Upon identification of the factual migraine triggers, said triggers are indicated by means of an output unit in form of a personalized feedback specific for the user at hand.

Accordingly, the present system and method provide the user the possibility to identify his/her personal migraine triggers by allocating a migraine episode to one or more detected potential migraine triggers, further valuable information about his/her factual migraine triggers and information in which way said triggers may be effectively prevented in future.

Thereby, the present system and method affords early detection (1-4 days before the attack) of impending onset of a migraine episode such that the migraine attack may be prevented. The interventions preventing the migraine are tailored to counter the personal triggers of the patient, which have been previously identified by the present system. Since the present system and method further affords identification as well as information about the situation/event of occurrence of the personal migraine triggers, the user may effectively avoid them in future. By tackling the identified triggers, migraine episodes are prevented. Furthermore, the present method and system provides a personalized tool for detecting and preventing a migraine episode due to considering the personal living conditions.

In contrast thereto, US 2003/0144829 A1 and US 2013/0066395 A1, which are mentioned in the introductory portion, both disclose the use of sensors for establishing a forecasting model affording the prediction of imminent onset of migraine. An early prediction of migraine onset, such as 1-4 days before the attack, is not afforded by these forecasting models. In particular, the documents are silent about using one or more previously identified signal characteristics in the brain activity signal indicative of an upcoming migraine attack for correlation with detected potential migraine triggers. In addition, both documents are silent about providing personalized feedback to the user informing about the determined one or more personal migraine triggers and/or how the user may prevent the determined one or more personal migraine triggers.

The expression “potential migraine triggers” as used herein refers to any circumstance(s) that may potentially cause(s) a migraine attack/episode in the user. Such potential migraine triggers may be found in a number of personal and external factors experienced by the user. Personal factors relate specifically to the user of the system and may be found in the user's personal condition, including for instance general health condition, age, stress and lifestyle habits, such as eating habits, and quality of sleep. For example, health status or condition of the user may be detected by means of a sensor or a combination of sensors, including for instance the heart rate or blood pressure of the user. External factors refer to sensory perception (by one or more of the “five senses”), including any environmental influence sensed by the user and which may be wither detected by means of a sensor or a combination of sensors or which may be (manually) entered in the migraine trigger detection unit.

The terms “correlations” and “correlating” as used herein refers to matching/assigning one or more identified signal characteristics in the brain activity signal to the detected potential migraine triggers.

Accordingly, “correlating” involves identifying a pattern within the potential migraine triggers which may give raise to a migraine attack. Such a pattern may involve the signal intensity of a potential migraine trigger, duration, regularity and occurrence of a signal of a single personal migraine trigger or a combination and interrelation thereof. These patterns are compared with the identified signal characteristics in the brain activity signal, i.e. the information that there is a forthcoming migraine attack. For this purpose the one or more identified signal characteristics and signal(s) from one or more sensors used for detecting potential migraine triggers may be transformed in the time-frequency domain. Each further correlation provides further information about the pattern of the potential migraine triggers, thereby permitting detecting of repeating patterns in the potential migraine triggers. Such an identified and repeated found pattern is indicative for one or more personal migraine triggers, i.e. migraine triggers causing with a high probability the onset of a migraine episode. Hence, the present system and method may be trained/taught by repeatedly monitoring the characteristics reflecting the onset of a migraine episode.

A “personal migraine trigger” as used herein refers to the identified pattern within the potential migraine triggers indicative with a high probability, such as at least 75%, at least 80%, at least 90%, at least 95% at least 99%, or 100% for a migraine attack. A “personal migraine trigger” may be therefore a personal migraine trigger that indeed or with a certain probability, such as e.g. at least 75%, causes a migraine attack in the user. It will be appreciated that each “correlation” performed, i.e. each comparison of potential migraine triggers to a repeated occurrence of migraine episodes, renders the identified personal migraine triggers more accurate in terms of their identity and possibly also their strength. A personal migraine trigger is the factual triggering event of a migraine episode which has been found for a particular person, i.e. a person/user using the brain activity sensor and the migraine trigger detection unit of the present system and method.

The expression “personalized feedback” as used herein refers to any information connected to the identified personal migraine triggers. This includes their nature, occurrence, strength and possible interaction with other potential migraine triggers and/or personal migraine triggers. The personalized feedback further informs the user about possibilties to prevent the determined one or more personal migraine triggers, thereby avoiding the onset of a new possible migraine episode and/or alleviating the symptoms thereof. In general, the personalized feedback provides personalized services to tackle the migraine triggers, thereby effectively eliminating and preventing the migraine onset altogether.

According to a preferred embodiment of the present invention the brain activity sensor includes an electroencephalography (EEG) sensor and the brain activity signal includes an EEG signal.

Any number of EEG sensors may be employed for determining the EEG signal. It is known that these sensors need to be positioned on special locations on the user's head. The number of EEG sensors may be one or more, such as 2-100, 5-50, 8-40, 10-30, 15-25 or 20 sensors. These sensors are allocated at those positions of the head which preferably afford the best possible detection of delta, theta and other waves and further the contingent negative variation (CNV) of the EEG signal. Alternatively, any suitable functional neuroimaging technique for detecting brain activity signals, particularly fNIRS or fMRI may be employed. These techniques are known to the skilled person.

According to another embodiment of the present invention the processing unit is configured to process the EEG signal to identify the one or more signal characteristics in the EEG signal by analyzing one or more of the delta and theta frequency bands of the EEG signal, the alpha waves of the EEG signal, and the contingent negative variation of the EEG signal.

These signal characteristics in the EEG signal encompass one or more peaks, strength/intensity of the peaks, and distance there between of the delta, theta and alpha waves of the EEG signal as well as the CNV of the EEG signal.

Processing of an EEG signal to identify the one or more signal characteristics in EEG signal, including delta, theta and other waves and further CNV of the EEG signal, is disclosed for example in Siniatchkin M. Gerber, W. D., Kropp P., Vein A.: How the brain anticipates an attack: A study of neurophysiological periodicity in migraine. Funct. Neurol. 1999 April-June; 14(2): 69-77 the content of which is incorporated by way of reference.

CNV detection may require the user to take a particular body posture or to perform a particular postural change. Respective information/instructions including information about the correct use/position of the EEG may be provided by the output unit. A number of other studies have demonstrated an increase in daily hassles, tension and depression during days leading up to a migraine attack. The presence of specific symptoms preceding the attack, sometimes occurring up to 48 h beforehand, suggests a slow development of the cascade of events leading to headache. A migraine attack can be predicted using EEG brain activity by: a significant increase in the power of delta and theta frequency bands, as well as in the asymmetry in alpha waves; and/or a significant increase in an early aspect of the EEG event related wave (called contingent negative variation, CNV amplitude). Migraine may be characterized by periodic CNV and EEG power spectrum changes during the pain-free interval. The abnormalities in cortical excitability and arousal are preferably observed before an attack (1-4 days before the attack) and could be used to predict the next migraine episode.

According to still another embodiment of the present invention the migraine trigger detection unit comprises one or more detection sensors for detecting one or more detection signals, and wherein the migraine trigger detection unit is configured to detect the potential migraine triggers by means of a signal analysis of the one or more detection signals.

The kind of the sensor as well as their number is not particularly limited. At least 1 sensor is preferably employed. It is possible to include two or more sensors of the same kind for providing improved signal quality and/or reliability. Each of the sensors employed provides one or more signal which may be subjected to signal processing such as signal transformation, for further use. Each of the detection signals provides information on time-dependent variation of signal intensity. Thereby, occurrence of a particular signal, which is derived from a particular sensor for measuring a potential migraine trigger, may be defined in dependence of time of occurrence. For instance, a light sensor may provide a detection signal giving information on light intensity and changes thereof in course of the day. Thereby, high light intensities as well as their duration and time of occurrence are provided. Time of occurrence enables the possibility to interfere the reason, if necessary. Such conversion in the time frequency domain of the signals affords easily comparison with the identified signal characteristics in the brain activity signal which are also in a time-frequency domain. This enables an easy correlation of the pattern of detected potential migraine triggers. Alternatively or in addition, thresholds may be used for identifying a signal of particular high intensity. In this way only signals with intensity above the threshold may be used for correlation with the one or more identified signal characteristics in the brain activity signal. A threshold may be used to remove a certain percentage of the signals detected by a particular sensor, such as at least 80%, at least 90%, at least 99%, or at least 99.99%. The threshold is preferably set in a manner permitting at least removal of background noise. Respective techniques are well known in the art.

According to an embodiment of the present the one or more detection sensor comprises a vital sign sensor for detecting a vital sign signal of the user, the vital sign sensor including one or more of: (i) a heart rate sensor, (ii) blood pressure sensor, (iii) a galvanic skin response sensor, and (iv) a photoplethysmographic (PPG) sensor.

The above-mentioned sensors are well known in the art. The sensors may be used alone or in combination. A possible field of application is stress monitoring. Stress may be monitored by measuring galvanic skin response and/or photoplethysmography. The heart rate and the blood pressure may provide further information regarding to stress. Stress may be detected by identifying significant increases in the galvanic skin response signal, the heart rate signal and a decreased amplitude of heart rate variability.

According to another embodiment of the present invention the one or more detection sensors comprises an environmental sensor for detecting an environmental influence in an environment of the user, the environmental sensor including one or more of: (i) a microphone, (ii) a light sensor, (iii) an olfactory sensor, (iv) a tactile sensor, (v) a temperature sensor, and (vi) a humidity sensor.

As indicated above, environmental influences encompass potentially each kind of sensual perception. Each of the sensors provides after transformation of the detection signal in the time-frequency domain information about the intensity/strength occurrence and time duration of the signal. The light sensor may be adapted enabling the detection of flashes and/or smokes. Changes in temperature and humidity may be either analyzed by using a temperature sensor or humidity sensor respectively or may be determined by analysis of failure trends in the signals acquisitioned by the sensors above.

According to another embodiment of the present invention the one or more detection sensors includes a sleep quality sensor for detecting a signal indicative for sleep quality of the user.

The signals indicative for sleep quality may be provided by an accelerometer that is arranged at or within the bed of the user. Alternatively, the signals indicative for sleep quality may be provided by pulse rate sensor or a photophletysmography (PPG) sensor. The PPG sensor is designated for enabling measuring the cardiac activity of the user. It may be used to determine accurately in—beat intervals an extract cardiac features required for sleep staging. The accelerometer sensor may provide a motion signal of the user. The accelerometer sensor employed is preferably a tri-axis accelerometer known in the art. In general, any information on the quality of sleep including information about the different sleep stages as well as their duration and succession may be provided. Quality of sleep may includes information of one or more of duration of sleep, amount of deep sleep, sleep fragmentation, number of arousals and REM onset detection. The user may also subjectively define the quality of sleep by answering a questionnaire provided by the output unit, e.g. the user is requested to enter 10 for very good sleep quality or 1 for bad sleep quality. The user's response may be an input in the input unit.

According to another embodiment of the present invention the migraine trigger detection unit comprises an input unit that enables the user to manually enter personalized information with at least one of a sleep quality of the user, dietary habits of the user and other incidents including potential migraine triggers, and wherein the migraine trigger detection unit is configured to detect the potential migraine triggers based in the manually entered personalized information.

The input unit is not particularly limited and may include tactile input means and input through spoken language. Tactile input means comprise for instance a keyboard, and input means of already existing devices, such as the keyboard of a computer, handheld devices, including tablets and smartphones. Accordingly, the user may enter information for instance in the smartphone which in turn is transmitted to the present system. Personalized information preferably encompasses information which may be not easily determined by use of one or more sensors. This comprises inter alia subjective information, such as the quality of sleep and wellness. Other information such as dietary habits of the user is also difficult to determine by means of a sensor and are therefore preferably entered manually. With respect to the dietary habitude a number of specific foods and/or beverages may be entered. Specific foods which may cause a migraine episode (alone or in combination with other events) and which may be input by the user include for instance one or more of ripened cheeses (such as Cheddar, Emmentaler, Stilton, Brie and Camembert), chocolate, marinated, pickled, or fermented food, foods that contain nitrites or nitrates (bacon, hot dogs) or mono-sodium glutamate (soy sauce, meat tenderizers, seasoned salt), sour cream, nuts, peanut butter, sourdough bread, broad beans, lima beans, fava beans, snow peas, figs, raisins, papayas, avocados, red plums, citrus fruits. In addition or alternatively, consuming of excessive amounts (such as more than 200 ml in total) of caffeinated beverages such as tea, coffee, or Coca-Cola®, alcohol (such as red vine and bear) may be input. Other possible dietary habits may be introduced by the user.

Other incidents including potential migraine triggers are for example incidents which may be found at the working place. Depending on the work performed, there may be exposure to different chemicals and/or electromagnetic radiation. This information may be entered manually in the system and subjected to correlation with one or more identified signal characteristics in the brain activity signal for identifying one or more correlation indicative of a factual migraine trigger.

According to another embodiment of the present invention the system further comprises a bracelet, chest strap and/or watch which comprises the migraine trigger detection unit.

The advantage of such a bracelet, chest strap and/or watch is that the user may wear the migraine trigger detection unit at any time and at essentially each kind of place. Accordingly, continuous monitoring of potential migraine triggers is rendered possible. The one or more sensors may be employed in form of an electronic wafer, i.e. the one or more sensors form part of a wafer. Alternatively, a modular system may be employed wherein the user may alter the configuration of the one ore more sensors employed in a migraine trigger detection unit according his/her personal needs. In case a potential migraine trigger has been verified as not being personal migraine trigger, the user may remove a respective sensor from the bracelet, chest strap and/or watch. This provides the possibility to connect another kind of sensor for testing. Alternatively or in addition, this offers the possibility to detect potential migraine triggers according to the user's life circumstances, such as the use of a first set of sensors for daily live and a second set for holiday.

According to another embodiment of the present invention the output unit comprises one or more of a display, a loudspeaker, and a tactile actuator.

Output units of the above mentioned kind are known in the art. The output unit may be in form of a wireless device remote from the system. For instance, the output unit for providing the user with a personalized feedback may be in form of an App on a smartphone and/or tablet computer. In such a case the output unit may also serve as an input unit.

In general the components of the present system including inter alia the brain activity sensor, migraine trigger detection unit, processing unit, output unit, input unit and feedback evaluation unit may be present in any number or interconnection. For instance, 1, 2, 3, 4, or 5 migraine trigger detection units may be used. This offers the possibility to take use of more sensors and/or positioning them in better suited location, such as chest or wrist. In addition or alternatively 1, 2, 3, 4, or 5 output units may be employed. One output unit may be used for providing the feedback information to the user, and another for providing the feedback information to a medical practitioner. It will be further appreciated that interconnection between each of brain activity sensor, migraine trigger detection unit, processing unit, output unit, input unit and feedback evaluation unit with any other component of the system may be wireless or wired. Combinations of wireless or wired interconnections may be used as well. Wireless or remote interconnection may be afforded by radio signals. It will be further appreciated that some of the components may be combined. For instance, using a watch which comprises the migraine trigger detection unit may also encompass using of the display and/or speakers of the watch as output unit and optional buttons of the watch as input unit. Using of a handheld device, in particular a smartphone or tablet computer, may encompass that the device's processor is used as processing unit, device's screen/speakers are used as output unit, and device's input means are used as input unit.

According to another embodiment of the present invention, the system further comprises a storage unit for storing the personalized feedback, and a feedback evaluation unit for evaluating an influence of personalized feedback on the user by comparing the personalized feedback stored in the storage unit with the brain activity signal and/or detected potential migraine triggers.

The feedback evaluation unit in general allows the user to follow up progress upon determining one or more personal migraine triggers. In addition, the user may compare and/or re-evaluate the stored personalized feedback in view of current personalized feedback. The feedback evaluation unit may provide the user the possibility for repeated performing a correlation between the already determined one or more personal migraine triggers in view of the brain activity signal for possibly identifying yet undiscovered relationship between personal migraine trigger and a forthcoming migraine episode. Alternatively or in addition, the stored personalized feedback may be also used for teaching the present system in terms of more accurate defining the personal migraine triggers by e.g. excluding false personal migraine triggers which have been found positive by chance. The feedback evaluation unit may further comprise a storage unit for storing the personalized feedback.

The feedback evaluation unit may keep monitoring the brain activity to see if indeed the trends indicative of impending migraine are disappearing (as the intervention is applied or after the intervention) or not. In line with this the processing unit of the present system may be configured not to suggest the patient and/or a third person, such as a medical caregiver, taking any measure in case trends indicative of impending migraine are disappearing. Hence, the processing unit may be configured not to give any advice in that case. To the contrary, if the trends indicative of impending migraine are (still) present, the processing unit of the present system may be configured to give a corresponding advice. Such an advice may encompass the suggestion to detect other potential migraine triggers by including corresponding sensors or inputting corresponding information.

According to an embodiment of the present invention, the feedback evaluation unit further comprises an internet module for sharing the stored personalized feedback.

The internet module provides the user the possibility for sharing or forwarding stored personalized feedback to any other person, such as a medical caregiver. The caregiver may than evaluate the information provided and make suggestions, including for instance any (medical) intervention and/or treatment regime, to the user. The forwarding of the stored personalized feedback may be performed automatically. Preferably, the stored personalized feedback is automatically shared upon an influence of the personalized feedback on the user is evaluated.

It will be readily understood that the above mentioned embodiments relating to a system or method for detecting and preventing a migraine episode may be applied likewise to a system or method for detecting and preventing an epilepsy seizure episode.

Epilepsy seizures often develop minutes to hours before clinical onset (Litt and Echauz, “Prediction of epileptic seizures”, The Lancet Neurology, Vol Nol, page 22-30, May 2002). Seizures in temporal-lobe epilepsy can be predicted at least 20 min before unequivocal electrographic onset and also substantial evidence that other changes that begin 1.0-1.5 h before onset in temporal-lobe epilepsy are highly associated with seizures and are predictive. There are other changes that occur up to a few hours before unequivocal EEG onset of seizures, such as bursts of long-term energy, changes in phase synchronisation, and focal subclinical seizures on EEG, which are associated with periods of increased probability of seizure onset. Abnormal activity in the hippocampus becomes correlated or “entrained” bilaterally in the 10-20 min before the unequivocal electrical onset of seizures in temporal-lobe epilepsy implying that the epileptic focus probably requires coincident activation of other brain regions—cortical, subcortical, or both—to generate clinical seizures.

According to an embodiment of the present invention, the onset of epilepsy may be detected and/or prevented at least one day before the unequivocal electrographic onset, preferably at least 18 hours, 12 hours, 6 hours, 3 hours, 2 hours 1.5 hours, 1 hour, 30 minutes or 20 minutes before the unequivocal electrographic onset.

Techniques which may be used to forecast seizures, i.e. for identifying one or more correlations between the one or more identified signal characteristics in the brain activity signal and the detected potential epilepsy triggers, include frequency-based methods, statistical analysis of EEG signals, non-linear dynamics (chaos), and intelligent engineered systems (Osorio et al, “Real Time automated detection and Quantitative Analysis of Seizures and short-term prediction of clinical onset. Epilepsia, 39(6):615-627, 1998”). These techniques are well known in the art and may be easily implemented.

According to a preferred embodiment, epileptic attacks are predicted by EEG brain activity. Frequency-domain analysis is used to decompose the EEG signal into components of different frequencies. For example, it has been found that bursts of activity in the range 15-25 Hz, start about 2 h before seizure onset in some patients with temporal-lobe epilepsy. These discharges change their frequency steadily so that they become faster or slower or some combination of the two over time—similar to the doppler effect heard when a train whistle or car horn passes by an observer. Such frequency can be associated with seizure patterns on the EEG.

The present invention affords identification of potential and factual epilepsy triggers. These triggers may be the same as for migraine indicated above. Preferred triggers are one or more of stress; environmental triggers: hazardous light stimuli such as flashes, flickering, bright lighting; one or more of the environmental triggers while being exposed exposed to TV, or another screen, such as a computer screen; low quality of sleep, including a change in sleep schedules; use of alcohol and/or drugs.

The herein presented system and method tests the brain activity of the user in order to determine the likelihood and time frame of a epilepsy seizure onset. This is performed by the brain activity sensor which detects and optionally records a brain activity signal, such as an electroencephalography (EEG) signal, functional near-infrared spectroscopy (fNIRS) or functional magnetic resonance imaging (fMRI). The brain activity signal encompasses characteristics reflecting the onset of a epilepsy seizure episode in closer future. Such characteristics are particular wave(s), wave form(s), peak(s), peak area(s) and peak envelope(s) within the brain activity signal indicating the development of migraine and/or epilepsy seizure in closer future.

The one or more signal characteristics in the brain activity signal that are indicative of an upcoming epileptic attack require detecting the brain activity signal for a rather short period of time and low repetition frequency.

In addition, the present system and method identifies potential epilepsy triggers including all circumstances that may potentially cause a epileptic attack in the user which may be found in the personal living conditions. These potential epilepsy triggers are monitored along the user's day to possibly cover each conceivable, yet unknown factual epilepsy trigger. Each of the detected potential epilepsy triggers is subjected, alone or in combination with one or more other potential epilepsy triggers, to a correlation with the characteristics in the brain activity signal. By correlation of the one or more identified signal characteristics in the brain activity signal and the detected potential epilepsy triggers, the system and method reveals the one or more personal epilepsy triggers, i.e. epilepsy triggers that do actually cause epilepsy in the user at hand.

Repeated correlation of signal characteristics obtained from additional measurements with already identified personal epilepsy triggers, further potential epilepsy triggers and potential epilepsy triggers which are suspected to be factual personal epilepsy triggers (i.e. unconfident personal epilepsy triggers wherein still further testing is required) permits teaching the system and method. Upon identification of the factual epilepsy triggers, said triggers are indicated by means of an output unit in form of a personalized feedback specific for the user at hand.

Accordingly, the present system and method provide the user the possibility to identify his/her personal epilepsy triggers by allocating a epilepsy seizure episode to one or more detected potential epilepsy triggers, further valuable information about his/her factual epilepsy triggers and information in which way said triggers may be effectively prevented in future.

Thereby, the present system and method affords early detection of impending onset of a epilepsy seizure episode such that the epileptic attack may be prevented. The interventions preventing the epilepsy are tailored to counter the personal triggers of the patient, which have been previously identified by the present system. Since the present system and method further affords identification as well as information about the situation/event of occurrence of the personal epilepsy triggers, the user may effectively avoid them in future. By tackling the identified triggers, epilepsy seizure episodes are prevented. Furthermore, the present method and system provides a personalized tool for detecting and preventing a epilepsy seizure episode due to considering the personal living conditions.

The expression “potential epilepsy triggers” as used herein refers to any circumstance(s) that may potentially cause(s) a epileptic attack/episode in the user. Such potential epilepsy triggers may be found in a number of personal and external factors experienced by the user. Personal factors relate specifically to the user of the system and may be found in the user's personal condition, including for instance general health condition, age, stress and lifestyle habits, such as eating habits, and quality of sleep. For example, health status or condition of the user may be detected by means of a sensor or a combination of sensors, including for instance the heart rate or blood pressure of the user. External factors refer to sensory perception (by one or more of the “five senses”), including any environmental influence sensed by the user and which may be wither detected by means of a sensor or a combination of sensors or which may be (manually) entered in the epilepsy trigger detection unit.

The terms “correlations” and “correlating” as used herein refers to matching/assigning one or more identified signal characteristics in the brain activity signal to the detected potential epilepsy triggers.

Accordingly, “correlating” involves identifying a pattern within the potential epilepsy triggers which may give raise to a epileptic attack. Such a pattern may involve the signal intensity of a potential epilepsy trigger, duration, regularity and occurrence of a signal of a single personal epilepsy trigger or a combination and interrelation thereof. These patterns are compared with the identified signal characteristics in the brain activity signal, i.e. the information that there is a forthcoming epileptic attack. For this purpose the one or more identified signal characteristics and signal(s) from one or more sensors used for detecting potential epilepsy triggers may be transformed in the time-frequency domain. Each further correlation provides further information about the pattern of the potential epilepsy triggers, thereby permitting detecting of repeating patterns in the potential epilepsy triggers. Such an identified and repeated found pattern is indicative for one or more personal epilepsy triggers, i.e. epilepsy triggers causing with a high probability the onset of a epilepsy seizure episode. Hence, the present system and method may be trained/taught by repeatedly monitoring the characteristics reflecting the onset of a epilepsy seizure episode.

A “personal epilepsy trigger” as used herein refers to the identified pattern within the potential epilepsy triggers indicative with a high probability, such as at least 75%, at least 80%, at least 90%, at least 95% at least 99%, or 100% for a epileptic attack. A “personal epilepsy trigger” may be therefore a personal epilepsy trigger that indeed or with a certain probability, such as e.g. at least 75%, causes a epileptic attack in the user. It will be appreciated that each “correlation” performed, i.e. each comparison of potential epilepsy triggers to a repeated occurrence of epilepsy seizure episodes, renders the identified personal epilepsy triggers more accurate in terms of their identity and possibly also their strength. A personal epilepsy trigger is the factual triggering event of a epilepsy seizure episode which has been found for a particular person, i.e. a person/user using the brain activity sensor and the epilepsy trigger detection unit of the present system and method.

The expression “personalized feedback” as used herein refers to any information connected to the identified personal epilepsy triggers. This includes their nature, occurrence, strength and possible interaction with other potential epilepsy triggers and/or personal epilepsy triggers. The personalized feedback further informs the user about possibilties to prevent the determined one or more personal epilepsy triggers, thereby avoiding the onset of a new possible epilepsy seizure episode and/or alleviating the symptoms thereof. In general, the personalized feedback provides personalized services to tackle the epilepsy triggers, thereby effectively eliminating and preventing the epilepsy onset altogether.

According to a preferred embodiment of the present invention the brain activity sensor includes an electroencephalography (EEG) sensor and the brain activity signal includes an EEG signal. Any number and kind of EEG sensors as indicated above may be employed. The sensors are allocated at those positions of the head which preferably afford the best possible detection of signals indicate for a possible epilepsy attack. Alternatively, any suitable functional neuroimaging technique for detecting brain activity signals, particularly fNIRS or fMRI may be employed. These techniques are known to the skilled person.

According to another embodiment of the present invention the processing unit is configured to process the EEG signal to identify the one or more signal characteristics in the EEG signal by analyzing different bands of the EEG signal, the alpha waves of the EEG signal, and the contingent negative variation of the EEG signal. These signal characteristics in the EEG signal encompass one or more peaks, strength/intensity of the peaks, and distance there between of the delta, theta and alpha waves of the EEG signal as well as the CNV of the EEG signaProcessing of an EEG signal to identify the one or more signal characteristics in EEG signal, including delta, theta and other waves and further CNV of the EEG signal, is disclosed for example in Siniatchkin M. Gerber, W. D., Kropp P., Vein A.: How the brain anticipates an attack: A study of neurophysiological periodicity in migraine. Funct. Neurol. 1999 April-June; 14(2): 69-77 the content of which is incorporated by way of reference.

CNV detection may require the user to take a particular body posture or to perform a particular postural change. Respective information/instructions including information about the correct use/position of the EEG may be provided by the output unit. A epileptic attack can be predicted using EEG brain activity by identifying triggers that significantly correlate with modified brain activity. Thes e triggers are identified as causing epilepsy seizure in the case of the patient at hand. EEG brain activity may encompass a significant increase and/or asymmetry in the power of specific signals and bands, such as delta and theta frequency bands. The abnormalities are preferably observed before an attack and could be used to predict the next epilepsy seizure episode.

According to still another embodiment of the present invention the epilepsy trigger detection unit comprises one or more detection sensors for detecting one or more detection signals, and wherein the epilepsy trigger detection unit is configured to detect the potential epilepsy triggers by means of a signal analysis of the one or more detection signals. The kind of the sensor as well as their number is not particularly limited and may be corresponding to the sensor types, their number and other sensor properties as indicated above for detection/prediction and/or prevention of migraine.

According to an embodiment of the present the one or more detection sensor comprises a vital sign sensor for detecting a vital sign signal of the user, the vital sign sensor including one or more of: (i) a heart rate sensor, (ii) blood pressure sensor, (iii) a galvanic skin response sensor, and (iv) a photoplethysmographic (PPG) sensor. Preferably, corresponding sensor types, number of sensors and other sensor properties as indicated above for detection/prediction and/or prevention of migraine may be employed.

The present system may afford automatic epilepsy seizure prediction by identifying trends indicative of approaching epilepsy seizure onset in the patient EEG brain activity. In addition or alternatively, automatic identification of one or more epilepsy seizure triggers may be provided by monitoring potential epilepsy seizure trigger(s) and correlating potential triggers occurrence with brain activity trends indicative of approaching epilepsy seizure onset. Triggers that significantly correlate with modified brain activity are identified as causing epilepsy seizure in the case of the patient at hand. In addition or alternatively, a possibility for feedback and/or prevention may be provided. Feedback may be in the form of information on: the likelihood and timeframe of developing a epilepsy seizure onset, the epilepsy seizure triggers that lead to epilepsy seizure onset in the case of the patient at hand and/or services that tackle these triggers in order to prevent epilepsy seizure. Prevention may be enabled by providing personalized services that tackle the epilepsy seizure triggers, eliminates them thereby effectively eliminating and preventing the epilepsy seizure onset altogether.

The above-mentioned sensors are well known in the art. The sensors may be used alone or in combination. A possible field of application is stress monitoring. Stress may be monitored by measuring galvanic skin response and/or photoplethysmography. The heart rate, heart rate variability and the blood pressure may provide further information regarding to stress. Stress may be detected by identifying significant increases in the galvanic skin response signal, the heart rate signal and a decreased amplitude of heart rate variability.

According to another embodiment of the present invention the one or more detection sensors comprises an environmental sensor for detecting an environmental influence in an environment of the user, the environmental sensor including one or more of: (i) a microphone, (ii) a light sensor, (iii) an olfactory sensor, (iv) a tactile sensor, (v) a temperature sensor, and (vi) a humidity sensor.

As indicated above, environmental influences encompass potentially each kind of sensual perception. Each of the sensors provides after transformation of the detection signal in the time-frequency domain information about the intensity/strength occurrence and time duration of the signal. The light sensor may be adapted enabling the detection of flashes and/or smokes. Changes in temperature and humidity may be either analyzed by using a temperature sensor or humidity sensor respectively or may be determined by analysis of failure trends in the signals acquisitioned by the sensors above.

According to another embodiment of the present invention the one or more detection sensors includes a sleep quality sensor for detecting a signal indicative for sleep quality of the user.

The signals indicative for sleep quality may be provided by an accelerometer that is arranged at or within the bed of the user. Alternatively, the signals indicative for sleep quality may be provided by pulse rate sensor or a photophletysmography (PPG) sensor. The PPG sensor is designated for enabling measuring the cardiac activity of the user. It may be used to determine accurately in—beat intervals an extract cardiac features required for sleep staging. The accelerometer sensor may provide a motion signal of the user. The accelerometer sensor employed is preferably a tri-axis accelerometer known in the art. In general, any information on the quality of sleep including information about the different sleep stages as well as their duration and succession may be provided. Quality of sleep may includes information of one or more of duration of sleep, amount of deep sleep, sleep fragmentation, number of arousals and REM onset detection. The user may also subjectively define the quality of sleep by answering a questionnaire provided by the output unit, e.g. the user is requested to enter 10 for very good sleep quality or 1 for bad sleep quality. The user's response may be an input in the input unit.

According to another embodiment of the present invention the epilepsy trigger detection unit comprises an input unit that enables the user to manually enter personalized information with at least one of a sleep quality of the user, dietary habits of the user and other incidents including potential epilepsy triggers, and wherein the epilepsy trigger detection unit is configured to detect the potential epilepsy triggers based in the manually entered personalized information.

The input unit is not particularly limited and may include tactile input means and input through spoken language. Tactile input means comprise for instance a keyboard, and input means of already existing devices, such as the keyboard of a computer, handheld devices, including tablets and smartphones. Accordingly, the user may enter information for instance in the smartphone which in turn is transmitted to the present system. Personalized information preferably encompasses information which may be not easily determined by use of one or more sensors. This comprises inter alia subjective information, such as the quality of sleep and wellness. Other information such as dietary habits of the user is also difficult to determine by means of a sensor and are therefore preferably entered manually. With respect to the dietary habitude a number of specific foods and/or beverages may be entered. In formation about specific foods which may cause a epilepsy seizure episode (alone or in combination with other events) and/or are at least suspected in doing so may be recorded. In addition or alternatively, consuming of excessive amounts (such as more than 200 ml in total) of caffeinated beverages such as tea, coffee, or Coca-Cola®, alcohol (such as red vine and bear) may be input. Other possible dietary habits may be introduced by the user. Information about consume of alcohol and/or drugs, including their kind, quantity and regularity of intake, may represent important information and may be therefore included as well.

Other incidents including potential epilepsy triggers are for example incidents which may be found at the working place. Depending on the work performed, there may be exposure to different chemicals and/or electromagnetic radiation. This information may be entered manually in the system and subjected to correlation with one or more identified signal characteristics in the brain activity signal for identifying one or more correlation indicative of a factual epilepsy trigger.

According to another embodiment of the present invention the system further comprises a bracelet, chest strap and/or watch which comprises the epilepsy trigger detection unit.

The advantage of such a bracelet, chest strap and/or watch is that the user may wear the epilepsy trigger detection unit at any time and at essentially each kind of place. Accordingly, continuous monitoring of potential epilepsy triggers is rendered possible. The one or more sensors may be employed in form of an electronic wafer, i.e. the one or more sensors form part of a wafer. Alternatively, a modular system may be employed wherein the user may alter the configuration of the one ore more sensors employed in a epilepsy trigger detection unit according his/her personal needs. In case a potential epilepsy trigger has been verified as not being personal epilepsy trigger, the user may remove a respective sensor from the bracelet, chest strap and/or watch. This provides the possibility to connect another kind of sensor for testing. Alternatively or in addition, this offers the possibility to detect potential epilepsy triggers according to the user's life circumstances, such as the use of a first set of sensors for daily live and a second set for holiday.

According to another embodiment of the present invention the output unit comprises one or more of a display, a loudspeaker, and a tactile actuator. The output unit may be in form of a wireless device remote from the system. For instance, the output unit for providing the user with a personalized feedback may be in form of an App on a smartphone and/or tablet computer. In such a case the output unit may also serve as an input unit.

In general the components of the present system including inter alia the brain activity sensor, epilepsy trigger detection unit, processing unit, output unit, input unit and feedback evaluation unit may be present in any number or interconnection. For instance, 1, 2, 3, 4, or 5 epilepsy trigger detection units may be used. This offers the possibility to take use of more sensors and/or positioning them in better suited location, such as chest or wrist. In addition or alternatively 1, 2, 3, 4, or 5 output units may be employed. One output unit may be used for providing the feedback information to the user, and another for providing the feedback information to a medical practitioner. It will be further appreciated that interconnection between each of brain activity sensor, epilepsy trigger detection unit, processing unit, output unit, input unit and feedback evaluation unit with any other component of the system may be wireless or wired. Combinations of wireless or wired interconnections may be used as well. Wireless or remote interconnection may be afforded by radio signals. It will be further appreciated that some of the components may be combined. For instance, using a watch which comprises the epilepsy trigger detection unit may also encompass using of the display and/or speakers of the watch as output unit and optional buttons of the watch as input unit. Using of a handheld device, in particular a smartphone or tablet computer, may encompass that the device's processor is used as processing unit, device's screen/speakers are used as output unit, and device's input means are used as input unit.

According to another embodiment of the present invention, the system further comprises a storage unit for storing the personalized feedback, and a feedback evaluation unit for evaluating an influence of personalized feedback on the user by comparing the personalized feedback stored in the storage unit with the brain activity signal and/or detected potential epilepsy triggers.

The feedback evaluation unit in general allows the user to follow up progress upon determining one or more personal epilepsy triggers. In addition, the user may compare and/or re-evaluate the stored personalized feedback in view of current personalized feedback. The feedback evaluation unit may provide the user the possibility for repeated performing a correlation between the already determined one or more personal epilepsy triggers in view of the brain activity signal for possibly identifying yet undiscovered relationship between personal epilepsy trigger and a forthcoming epilepsy episode. Alternatively or in addition, the stored personalized feedback may be also used for teaching the present system in terms of more accurate defining the personal epilepsy triggers by e.g. excluding false personal epilepsy triggers which have been found positive by chance. The feedback evaluation unit may further comprise a storage unit for storing the personalized feedback.

The feedback evaluation unit may keep monitoring the brain activity to see if indeed the trends indicative of impending epilepsy are disappearing (as the intervention is applied or after the intervention) or not. In line with this the processing unit of the present system may be configured not to suggest the patient and/or a third person, such as a medical caregiver, taking any measure in case trends indicative of impending epilepsy are disappearing. Hence, the processing unit may be configured not to give any advice in that case. To the contrary, if the trends indicative of impending epilepsy are (still) present, the processing unit of the present system may be configured to give a corresponding advice. Such an advice may encompass the suggestion to detect other potential epilepsy triggers by including corresponding sensors or inputting corresponding information.

According to an embodiment of the present invention, the feedback evaluation unit further comprises an internet module for sharing the stored personalized feedback.

The internet module provides the user the possibility for sharing or forwarding stored personalized feedback to any other person, such as a medical caregiver. The caregiver may than evaluate the information provided and make suggestions, including for instance any (medical) intervention and/or treatment regime, to the user. The forwarding of the stored personalized feedback may be performed automatically. Preferably, the stored personalized feedback is automatically shared upon an influence of the personalized feedback on the user is evaluated.

According to a preferred embodiment of the present invention the epilepsy seizure episode arises from temporal-lobe epilepsy. Other ailments giving raise to an epilepsy seizure episode may encompass brain injury, stroke, brain tumors, and substance use disorders, including for instance alcohol and/or drugs. Known genetic mutations may be directly linked to some cases of epilepsy.

The following clauses section indicate embodiments directed to a system, method and computer program for detecting/predicting and preventing a epilepsy seizure episode in a user.

Clause 1: System for detecting and preventing a epilepsy seizure episode in a user, comprising:

a brain activity sensor for detecting a brain activity signal;

a epilepsy trigger detection unit for detecting potential epilepsy triggers, said potential epilepsy triggers being circumstances that may potentially cause a epileptic attack in the user;

a processing unit configured to:

(i) process the brain activity signal to identify one or more signal characteristics in the brain activity signal that are indicative of an upcoming epileptic attack in the user;

(ii) identify one or more correlations between the one or more identified signal characteristics in the brain activity signal and the detected potential epilepsy triggers;

(iii) determine, based on the identified one or more correlations, one or more personal epilepsy triggers that are likely to cause a epileptic attack in the user;

an output unit for providing the user with a personalized feedback informing the user about the determined one or more personal epilepsy triggers and/or how the user may prevent the determined one or more personal epilepsy triggers.

Clause 2: System of clause 1, wherein the brain activity sensor includes an EEG sensor and the brain activity signal includes an EEG signal.
Clause 3: System of clause 2, wherein the processing unit is configured to process the EEG signal to identify the one or more signal characteristics in the EEG signal by analysing one or more of the delta and theta frequency bands of the EEG signal, (ii) the alpha waves of the EEG signal, and (iii) the contingent negative variation of the EEG signal.
Clause 4: System of clause 1, wherein the epilepsy trigger detection unit comprises one or more detection sensors for detecting one or more detection signals, and wherein the epilepsy trigger detection unit is configured to detect the potential epilepsy triggers by means of a signal analysis of the one or more detection signals.
Clause 5: System of clause 4, wherein the one or more detection sensors comprises a vital sign sensor for detecting a vital sign signal of the user, the vital sign sensor including one or more of: (i) a heart rate sensor, (ii) a blood pressure sensor, (iii) a galvanic skin response sensor, and (iv) a photoplethysmographic sensor.
Clause 6: System of clause 4, wherein the one or more detection sensors comprises an environmental sensor for detecting an environmental influence in the environment of the user, the environmental sensor including one or more of: (i) a microphone, (ii) a light sensor, (iii) an olfactory sensor, (iv) a tactile sensor, (v) a temperature sensor, and (vi) a humidity sensor.
Clause 7: System of clause 4, wherein the one or more detection sensors include a sleep quality sensor for detecting a signal indicative of a sleep quality of the user.
Clause 8: System of clause 4, wherein the epilepsy trigger detection unit comprises an input unit that enables the user to manually enter personalized information on at least one of (i) a sleep quality of the user, (ii) dietary habits of the user, and (iii) other incidents including potential epilepsy triggers, and wherein the epilepsy trigger detection unit is configured to detect the potential epilepsy triggers based on the manually entered personalized information.
Clause 9: System of clause 1, further comprising a bracelet, chest strap and/or watch which comprises the epilepsy trigger detection unit.
Clause 10: System of clause 1, further comprising a headset or headband comprising the brain activity sensor.
Clause 11: System of clause 1, wherein the output unit comprises one or more of (i) a display, (ii) a loudspeaker, and (iii) a tactile actuator.
Clause 12: System of clause 1, further comprising a storage unit for storing the personalized feedback, and a feedback evaluation unit for evaluating an influence of personalized feedback on the user by comparing the personalized feedback stored in the storage unit with the brain activity signal and/or detected potential epilepsy triggers.
Clause 13: System of clause 12, wherein the feedback evaluation unit further comprises an internet module for sharing the stored personalized feedback.
Clause 14: Method for detecting and preventing a epilepsy seizure episode in a user, comprising the steps of:

detecting a brain activity signal of a user;

processing the brain activity signal to identify one or more signal characteristics in the brain activity signal that are indicative of an upcoming epileptic attack in the user;

detecting potential epilepsy triggers, said potential epilepsy triggers being circumstances that may potentially cause a epileptic attack in the user;

identifying one or more correlations between the one or more identified signal characteristics in the brain activity signal and the detected potential epilepsy triggers;

determining, based on the identified one or more correlations, one or more personal epilepsy triggers that are likely to cause a epileptic attack in the user;

providing the user with a personalized feedback informing the user about the determined one or more personal epilepsy triggers and/or how the user may prevent the epilepsy onset by countering the determined one or more personal epilepsy triggers.

Clause 15: Computer program comprising program code means for causing a computer to carry out the steps of the method as indicated in clause 14 when said computer program is carried out on a computer. These steps preferably include processing the brain activity signal, detecting potential epilepsy triggers, identifying one or more correlations, determining one or more personal epilepsy triggers, and providing the user with a personalized feedback.

Preferred embodiments of the invention are defined in the dependent claims. It shall be understood that the claimed method has similar and/or identical preferred embodiments as the claimed device and as defined in the dependent claims.

### BRIEF DESCRIPTION OF THE DRAWINGS

These and other aspects of the invention will be apparent from and elucidated with reference to the embodiments described hereinafter. In the following drawings

FIG. 1 schematically shows a system according to an embodiment of the present invention;

FIG. 2a shows an enlarged view of an exemplary headband comprising a brain activity sensor for use in the system according to the present invention;

FIG. 2b shows an enlarged view of an exemplary migraine trigger detection unit or epilepsy trigger detection unit embodied in form of a bracelet for use in the system according to the present invention;

FIG. 3 shows a diagram indicating signal intensities of a brain activity signal and detected potential migraine triggers; and

FIG. 4 shows a schematic block diagram illustrating the components of the system according to an embodiment of the present invention.

### DETAILED DESCRIPTION OF THE INVENTION

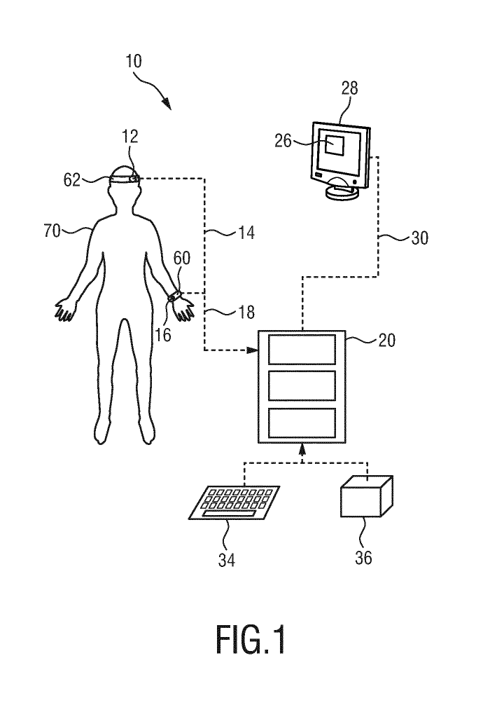

FIG. 1 schematically shows an embodiment of the system according to the present invention. The system is therein in its entirety denoted by reference numeral 10.

A person 70 wears a brain activity sensor 12 for detecting a brain activity signal 14 and a migraine trigger detection unit 16 for detecting potential migraine triggers 18. Brain activity signal 14 and detected potential migraine triggers 18 are transmitted to a processing unit 20. The processing unit 20 processes the brain activity signal 14 to identify one or more signal characteristics in the brain activity signal 14 that are indicative of an upcoming migraine attack in the user 70. The processing unit 20 further identifies one or more correlations between the one or more identified signal characteristics in the brain activity signal 14 and the detected potential migraine triggers 18 and determines, based on the identified one or more correlations, one or more personal migraine triggers that are likely to cause a migraine attack in the user 70, i.e. indeed or with a certain probability cause a migraine attack in the user 70.

The system 10 further comprises an output unit 28 for showing personalized feedback 26 which informs the user 70 about one or more personal migraine triggers and/or information how the user may prevent the determined one or more personal migraine triggers.

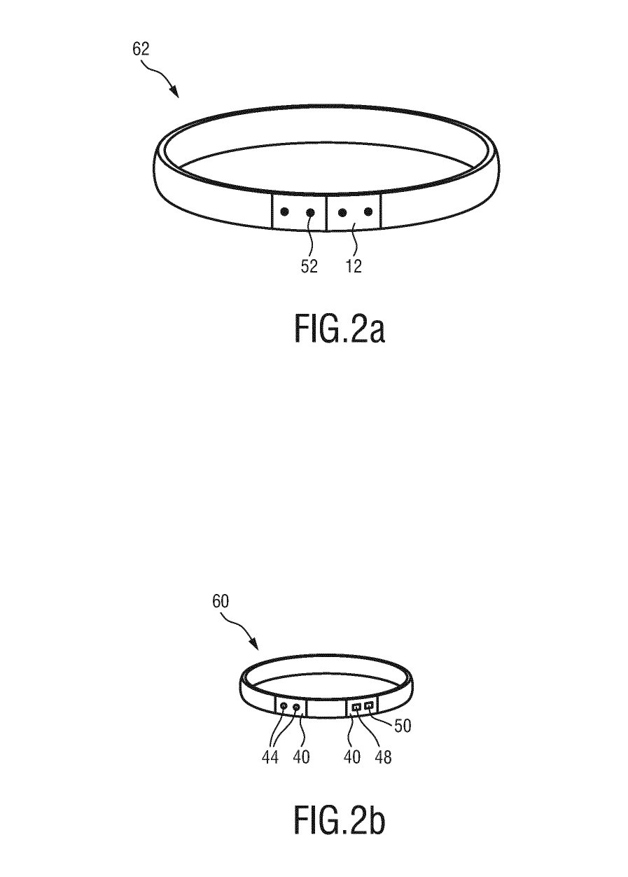

In the system 10, the brain activity sensor 12 for detecting a brain activity signal 14 identifies signals of approaching migraine onset in a user EEG brain activity. Brain activity is recorded on daily basis via a headband 62 which provides EEG measurements that last a matter of minutes, such as five minutes. An exemplary headband 62 is schematically shown in FIG. 2a. The user 70 may be instructed by the output unit 28 in which way the brain activity sensor 12 needs to be positioned on the user's head. The headband 62 employs a plurality of EEG sensors 52 providing the EEG signal 32. The EEG signals 32 are recorded and stored in the brain activity sensor 12 and transmitted to the processing unit 20 at a later stage.

The migraine trigger detection unit 16 is configured to detect potential migraine triggers in an automatic manner. This component 16 monitors possible migraine triggers and transmits respective signals to the processing unit 20 for further evaluation. The potential migraine triggers detected by the migraine trigger detection unit 16 encompass all potential migraine triggers, including stress, environmental influences, such as flashes, bright lights, smoke, loud noises, changes in temperature and humidity, quality of sleep and dietary habits and hydration. The migraine trigger detection unit 16 may thereto comprise a plurality of different detection sensors 40 which may be e.g. embodied in a bracelet 60. An exemplary bracelet 60 is schematically shown in FIG. 2b. The sensors 40 may include a vital sign sensor 44, such as e.g. a galvanic skin response sensor and a photoplethysmographic sensor for measuring a galvanic skin response signal, a heart rate variety signal and a heart rate signal. These signals are used for identifying/detecting stress by identifying significant increases in the galvanic skin response signal and the heart rate signal, as well as decreased amplitude of the heart rate variety signal. The galvanic skin response sensor and the photoplethysmographic sensor may be embedded on an electronic wafer to be used as add-on to a bracelet or watch 60.

Sensors 48 for measuring environmental influences may be included as well in the bracelet 60. Examples for such sensors 48 are: a microphone that detects noises, a light sensor that detects flashes and smoke, a temperature sensor that detects changes in temperature and a humidity sensor that detects changes in humidity. These sensors 48 may be also embedded on an electronic wafer to be used as add-on to the watch or bracelet 60 worn by user 70.

The quality of sleep of the user 70 may be additionally detected by use of a sleep quality sensor 50 included in the migraine trigger detection unit 16 for detecting a respective signal and providing the same to the processing unit 20. Accordingly, automatically detection of sleep quality is encompassed. Existing solutions monitor quality of sleep in brain activity, i.e. by using the signals 14 provided by a brain activity sensor 12 or employing already existing device, such as MyZeo by synthenet.

In addition, the user 70 may manually enter in the input unit 34 personalized information on the sleep quality in the migraine trigger detection unit. This is performed by a questionnaire presented to the user 70 and which may be completed each morning.

The input unit 34 furthermore enables the user 70 to enter dietary habits and information about hydration in the migraine trigger detection unit 16. The user 70 may select a respective menu shown on the output unit 28 and add information about missed, delayed or irregular meals and dehydration which may also give raise to a migraine attack.

The input unit 34 furthermore permits including of potential migraine triggers 18 for offering the user 70 the option to manually lock potential migraine triggers they have been exposed to. These potential migraine triggers include perfumes. The user 70 is asked if there has been exposure to strong smells and/or perfumes. A respective incident including time information may be entered in the migraine trigger detection unit 16.

Furthermore a list of particular dietary products known to potentially give raise to migraine attacks may be checked in form of a questionnaire. In addition, the time or time period of consuming said dietary product is entered by the user. Thereby, the information provided to the migraine trigger detection unit 16 comprises information on the identity of the potential migraine trigger 18 as well as a time signal indicative of the contact of the user 70 with said potential migraine trigger 18.

In the migraine trigger detection unit 16 furthermore information about user history are encompassed with prioritized common personal migraine triggers to restrict identification of one or more correlations only to the most likely potential migraine triggers for the particular user 70.

The processing unit 20 receives the brain activity signal 14 and all information reflecting potential migraine triggers 18 provided by the migraine trigger detection unit 16.

In particular, the processing unit 20 receives and processes the EEG signals 32 provided by the brain activity sensor 12 to identify signal characteristics 22 indicative of an upcoming migraine attack. A significant increase in the power of delta and theta frequency bands, in the alpha asymmetry and a significant increase in the early CNV amplitude during the pain-free interval (approx. 1-4 days before the migraine attack) is indicative for a forthcoming migraine episode. The processing unit 20 detects respective peaks and waveforms of high intensity.

In an optional step the processing unit 20 subjects the individual signals provided by the different detection sensors 40 and information entered in the input unit 34 (which are all in the time frequency-domain) to noise reduction to reduce noise to an acceptable level. The signals may be subjected to a threshold operation removing a certain percentage (such as 90%) of all signals with lower intensity.

The processing unit 20 correlates the signals reflecting potential migraine triggers 18 with the signal characteristics 22 indicative of an upcoming migraine attack. In particular, the processing unit 20 correlates potential migraine trigger occurrences with brain activity trends indicative of approaching migraine onset. Potential migraine triggers 18 that significantly (and repeatedly) correlate with modified brain activity are identified to determine, based on the identified one or more correlations (24), one or more personal migraine triggers 26 that indeed cause a migraine attack in the user (70) at hand. Hence, the system 10 learns and identifies the personal migraine triggers 26 by correlating the brain activity signals indicative of migraine susceptibility with the potential migraine triggers monitored.

Output unit 28 provides the user 70 with a personalized feedback 26 in view of the determined one or more personal migraine triggers 26. The personalized feedback 26 comprises general information about the determined one or more personal migraine triggers 26 and is further provided in the form of information on the likelihood and time frame of a developing a migraine onset and/or the migraine triggers that lead to migraine onset in the case of the user 70 at hand. The personalized feedback 26 furthermore provides services that tackle these triggers in order to prevent migraine onset in the particular user 70. Hence, the system 10 provides personalized services that tackle the personal migraine triggers 26, eliminates them thereby effectively eliminating and preventing the migraine onset altogether. The output unit 28 recommends and provides a relaxation service (e.g. embodied as closed loop relaxation service in case the personal migraine trigger 26 identified is stress). In case the personal migraine trigger 26 is an environmental influence, the output unit 28 informs the user 70 whenever environment includes said trigger and advises leaving environment for another suggested environment that fulfills the same function. If the personal migraine trigger 26 is a lack of quality of sleep, the output unit 28 provides relaxation techniques for improving sleep quality and optionally informs a caregiver in case the condition does not improve and further treatment is required. In case the personal migraine trigger 26 resides in dietary habits and possibly hydration, the output unit 28 may inform a caregiver with reminders when a user 70 requires support with eating and drinking. Output unit 28 also informs caregiver of foods and drinks that are personal migraine triggers 26 for the particular user 70 in order to be avoided.

FIG. 2a shows an enlarged view of the headband 62 comprising brain activity sensors 18 in form of EEG sensors 52 of FIG. 1. In the case shown the EEG sensors 52 are arranged in a manner that they are located on a forehead region (including temples) of the user 70. The headband 62 stores the of EEG sensors 52 and remotely transmits them to the processing unit 20.

FIG. 2b shows an enlarged view of a migraine trigger detection unit 16 of FIG. 1. The migraine trigger detection unit 16 is in form of a bracelet 60 including the detection sensors 40. The detection sensors 40 are provided by a clip-lock mechanism on the bracelet 60 permitting their individual release/removal for exchanging against other detection sensors 40 according to the user's 70 need and/or as suggested in the personalized feedback 30. The bracelet 62 is connected via processor to input unit 34 permitting manually entering and storing of personalized information.

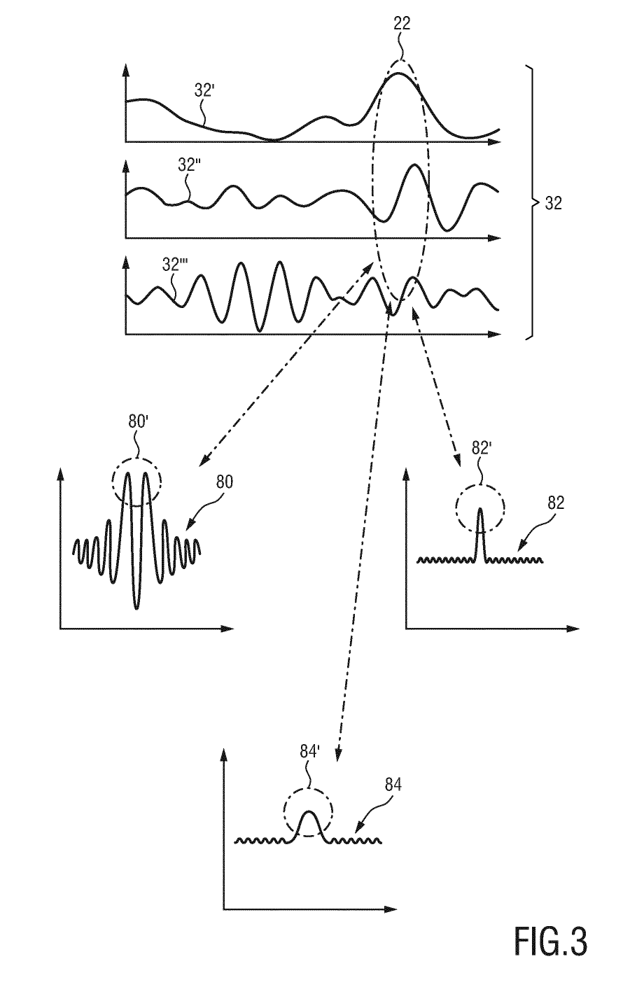

FIG. 3 shows an example of the signals processed by processing unit 20. EEG signals 32 comprise in this example information on delta waves 32′, theta waves 32″ and alpha waves 32′″. The EEG signals 32 are provided in the time-frequency domain (the ordinate showing the time in seconds). The delta waves 32′, theta waves 32″ and alpha waves 32′″ are evaluated to identify particular signal characteristics 22 which are indicative of a forthcoming migraine attack in the user 70. By way of example, acoustic signal 80, optical signal 82 and temperature signal 84 received by a microphone, light sensor and temperature sensor, respectively, are shown. The signals 80, 82 and 84 are shown in the time-frequency domain. It is clear that the time and intensity scale of the signals 80, 82 and 84 do not correspond to that of the delta waves 32′, theta waves 32″ and alpha waves 32′″. A particular loud noise in the acoustic signal 80 is shown as peaks 80′, whereas signals indicating light flashes 82′ and a signal indicating a higher temperature 84′ may be not only identified but also allocated to a particular time of the day and in turn to a specific event. The signals 80, 82 and 80 are subjected to filtering techniques including for example threshold values to identify relevant potential migraine triggers and to avoid background noise. Hence, only information about signals 80′, 82′ and 84′, either a specific peak of particular high intensity and/or several peaks of high intensity, are employed for correlating with the determined signal characteristics 22 in the delta waves 32′, theta waves 32″ and alpha waves 32′″. This correlation (indicated by arrows) permits identification of the one or more personal migraine triggers.

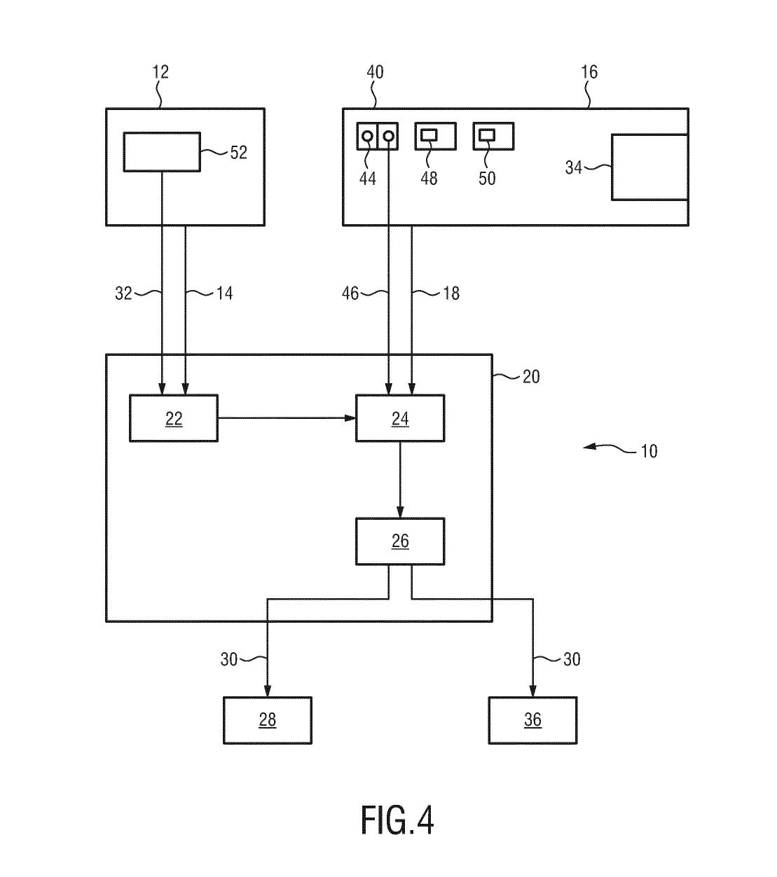

FIG. 4 shows a schematic block diagram illustrating the components of the system 10. Brain activity sensor 12 detects a brain signal 18. This may be performed by taking use of several EEG sensors 52 for detecting an EEG signal 32. Migraine trigger detection unit 16 includes one or more detection sensors 40. These sensors 40 comprise vital sign sensor 44 for detecting a vital sign signal 46, and environmental sensor 48 for detecting an environmental influence on the user 70 and a sleep quality sensor 50 for detecting a signal indicative of sleep quality of the user 70. The signals 46, 18 are provided to processing unit 20. Processing unit 20 in a first step identifies signal characteristics 22 in a brain activity signal 14 indicative of an upcoming migraine attack in the user 70. These signal characteristics are furthermore correlated with the detected potential migraine trigger 18 and 46. As indicated above this may performed by the occurrence of respective peaks, including intensity and position with respect to other signals, to find matching patterns indicative for a relationship between the detected potential migraine triggers 18, 46 and the one or more identified signal characteristics 22. Processing unit 20 furthermore determines, based on identified one or more correlations 24, one or more personal migraine triggers 26 causing a migraine attack in the user 70. The information on these personal migraine triggers 26 is furthermore used for providing the user with a personalized feedback on output unit 28. Additionally a personalized feedback is provided to a feedback evaluation unit 36 permitting the user assessment of the personalized feedback 30 in view of the brain activity signal and/or detected potential migraine triggers 18. The migraine trigger detection unit 16 furthermore comprises input unit 34 enabling the user 70 to enter personalized information.

An exemplary system for detecting/predicting and preventing an epilepsy seizure episode may test on daily basis (a few minutes, such as five minutes, four minutes, three minutes, two minutes, or one minute) the brain activity of the patient in order to determine the likelihood and timeframe of a epilepsy seizure onset. The system, including its required and optional components, may correspond to the above mentioned system for detection/prediction and denoted by reference numeral 10 and is shown in FIGS. 1, 2, and 4.

The user 70 wears the device that monitors and detects the user exposure to known potential epilepsy seizure triggers. The system learns and identifies the patient epilepsy seizure triggers by correlating the brain activity indicative of epilepsy seizure susceptibility with the triggers monitored. The system informs the user of epilepsy seizure susceptibility and triggers that cause it, as well as of services that tackle triggers and prevent the epilepsy seizure. A person 70 wears a brain activity sensor 12 for detecting a brain activity signal 14 and a epilepsy trigger detection unit 16 for detecting potential epilepsy triggers 18. Brain activity signal 14 and detected potential epilepsy triggers 18 are transmitted to a processing unit 20. The processing unit 20 processes the brain activity signal 14 to identify one or more signal characteristics in the brain activity signal 14 that are indicative of an upcoming epilepsy seizure attack in the user 70. The processing unit 20 further identifies one or more correlations between the one or more identified signal characteristics in the brain activity signal 14 and the detected potential epilepsy triggers 18 and determines, based on the identified one or more correlations, one or more personal epilepsy triggers that are likely to cause an epileptic attack in the user 70, i.e. indeed or with a certain probability cause an epileptic attack in the user 70. An output unit 28 may show personalized feedback 26 which informs the user 70 about one or more personal epilepsy triggers, i.e. essentially the identity of the one or more personal epilepsy triggers, and/or information how the user may prevent the determined one or more personal epilepsy triggers. The identified personal epilepsy trigger may be for example stress in that case the feedback 26 encompasses the information that the epilepsy trigger for the particular user 70 is stress and optionally supports the patient to de-stress/relax. This support may be for instance by providing respective information, instructions to exercise and/or contact with a clinician.

In the system 10, the brain activity sensor 12 for detecting a brain activity signal 14 identifies signals of approaching epilepsy onset in a user EEG brain activity. Brain activity is recorded on daily basis via a headband or headset 62 which provides EEG measurements that last a matter of minutes, such as five minutes. An exemplary headband 62 is schematically shown in FIG. 2a. The user 70 may be instructed by the output unit 28 in which way the brain activity sensor 12 needs to be positioned on the user's head. The headband 62 employs a plurality of EEG sensors 52 providing the EEG signal 32. The EEG signals 32 are recorded and stored in the brain activity sensor 12 and transmitted to the processing unit 20 at a later stage.

The epilepsy trigger detection unit 16 is configured to detect potential epilepsy triggers in an automatic manner. This component 16 monitors possible epilepsy triggers and transmits respective signals to the processing unit 20 for further evaluation. The potential epilepsy triggers detected by the epilepsy trigger detection unit 16 encompass all potential epilepsy triggers, including stress, environmental influences, such as flashes, bright lights, smoke, loud noises, changes in temperature and humidity, quality of sleep and dietary habits and hydration. The epilepsy trigger detection unit 16 may thereto comprise a plurality of different detection sensors 40 which may be e.g. embodied in a bracelet 60. An exemplary bracelet 60 is schematically shown in FIG. 2b. The sensors 40 may include a vital sign sensor 44, such as e.g. a galvanic skin response sensor and a photoplethysmographic sensor for measuring a galvanic skin response signal, a heart rate variety signal and a heart rate signal. These signals are used for identifying/detecting stress by identifying significant increases in the galvanic skin response signal and the heart rate signal, as well as decreased amplitude of the heart rate variety signal. The galvanic skin response sensor and the photoplethysmographic sensor may be embedded on an electronic wafer to be used as add-on to a bracelet or watch 60.

Sensors 48 for measuring environmental influences may be included as well in the bracelet 60. Examples for such sensors 48 are environmental sensors including one or more of a microphone that detects noises, a light sensor that detects hazardous light stimuli, such as flashes, flickering, bright lighting (which may be of particular impact for patients typically exposed to these while exposed to TV, or computer screen), a temperature sensor that detects changes in temperature and a humidity sensor that detects changes in humidity. Light sensors may for detect inadequate light intensity via analysis of value trends in the signals acquisitioned by different sensors, including one or more sensors employed for stress monitoring.

Different sensors 48 may be also combined for detection of rather complex epilepsy seizure triggers, such as stress. Stress monitoring may be peformed by galvanic skin response sensors and/or photo-pletismography sensors. Monitored signals may include galvanic skin response, heart rate variability and/or heart rate. Stress may be detected by identifying significant increases in galvanic skin response signal, and heart rate signal, as well as decreased amplitude of, heart rate variability.

Any combination of these sensors 48 may be also embedded on an electronic wafer to be used as add-on to the watch or bracelet 60 worn by user 70.

The quality of sleep of the user 70 may be additionally detected by use of a sleep quality sensor 50 included in the epilepsy trigger detection unit 16 for detecting a respective signal and providing the same to the processing unit 20. Accordingly, automatically detection of sleep quality is encompassed. Existing solutions monitor quality of sleep in brain activity, i.e. by using the signals 14 provided by a brain activity sensor 12 or employing already existing device, such as MyZeo by synthenet.

In addition, the user 70 may manually enter in the input unit 34 personalized information on the sleep quality in the epilepsy trigger detection unit. This is performed by a questionnaire presented to the user 70 and which may be completed each morning.

The input unit 34 furthermore enables the user 70 to enter dietary habits and information about hydration in the epilepsy trigger detection unit 16. The user 70 may select a respective menu shown on the output unit 28 and add information about missed, delayed or irregular meals and dehydration which may also give raise to a epilepsy attack.

The input unit 34 furthermore permits including of potential epilepsy triggers 18 for offering the user 70 the option to manually lock potential epilepsy triggers they have been exposed to. These potential epilepsy triggers include perfumes. The user 70 is asked if there has been exposure to strong smells and/or perfumes. A respective incident including time information may be entered in the epilepsy trigger detection unit 16.

Furthermore a list of particular dietary products known to potentially give raise to epilepsy attacks may be checked in form of a questionnaire, such as alcohol and/or drugs. In addition, the time or time period of consuming said dietary product is entered by the user. Thereby, the information provided to the epilepsy trigger detection unit 16 comprises information on the identity of the potential epilepsy trigger 18 as well as a time signal indicative of the contact of the user 70 with said potential epilepsy trigger 18.

In the epilepsy trigger detection unit 16 furthermore information about user history are encompassed with prioritized common personal epilepsy triggers to restrict identification of one or more correlations only to the most likely potential epilepsy triggers for the particular user 70. An in time system may use patient history with prioritized common personalized triggers to restrict monitoring only to those most likely triggers for this patient.

The processing unit 20 receives the brain activity signal 14 and all information reflecting potential epilepsy triggers 18 provided by the epilepsy trigger detection unit 16. In particular, the processing unit 20 receives and processes the EEG signals 32 provided by the brain activity sensor 12 to identify signal characteristics 22 indicative of an upcoming epilepsy attack. The processing unit 20 detects respective peaks and waveforms of high intensity. An attack is predicted by EEG brain activity. Frequency-domain analysis is used to decompose the EEG signal into components of different frequencies. Bursts of activity in the range 15-25 Hz, may start about 2 h before seizure onset in some patients with temporal-lobe epilepsy. These discharges change their frequency steadily so that they become faster or slower or some combination of the two over time—similar to the doppler effect heard when a train whistle or car horn passes by an observer. Such frequency is associated with seizure patterns on the EEG.

In an optional step the processing unit 20 subjects the individual signals provided by the different detection sensors 40 and information entered in the input unit 34 (which are all in the time frequency-domain) to noise reduction to reduce noise to an acceptable level. The signals may be subjected to a threshold operation removing a certain percentage (such as 90%) of all signals with lower intensity.

The processing unit 20 correlates the signals reflecting potential epilepsy triggers 18 with the signal characteristics 22 indicative of an upcoming epilepsy attack.

In particular, the processing unit 20 correlates potential epilepsy trigger occurrences with brain activity trends indicative of approaching epilepsy onset. Potential epilepsy triggers 18 that significantly (and repeatedly) correlate with modified brain activity are identified to determine, based on the identified one or more correlations (24), one or more personal epilepsy triggers 26 that indeed cause a epilepsy attack in the user (70) at hand. Hence, the system 10 learns and identifies the personal epilepsy triggers 26 by correlating the brain activity signals indicative of epilepsy susceptibility with the potential epilepsy triggers monitored.

Output unit 28 provides the user 70 with a personalized feedback 26 in view of the determined one or more personal epilepsy triggers 26. The personalized feedback 26 comprises general information about the determined one or more personal epilepsy triggers 26 and is further provided in the form of information on the likelihood and time frame of a developing a epilepsy onset and/or the epilepsy triggers that lead to epilepsy onset in the case of the user 70 at hand. The personalized feedback 26 furthermore provides services that tackle these triggers in order to prevent epilepsy onset in the particular user 70. Hence, the system 10 provides personalized services that tackle the personal epilepsy triggers 26, eliminates them thereby effectively eliminating and preventing the epilepsy onset altogether. The output unit 28 recommends and provides a relaxation service (e.g. embodied as closed loop relaxation service in case the personal epilepsy trigger 26 identified is stress). In case the personal epilepsy trigger 26 is an environmental influence, the output unit 28 informs the user 70 whenever environment includes said trigger and advises leaving environment for another suggested environment that fulfills the same function. System will inform user whenever environment includes trigger and advises leaving environment for another suggested environment that fulfils same function. If the personal epilepsy trigger 26 is a lack of quality of sleep, the output unit 28 provides relaxation techniques for improving sleep quality and optionally informs a caregiver in case the condition does not improve and further treatment is required. In case the personal epilepsy trigger 26 resides in dietary habits and possibly hydration, the output unit 28 may inform a caregiver with reminders when a user 70 requires support with eating and drinking. Output unit 28 also informs caregiver of foods and drinks that are personal epilepsy triggers 26 for the particular user 70 in order to be avoided.

Compared to similar systems of the prior art, the herein presented system in summary provides the following advantages: It enables a pro-active prediction of migraine onsets and/or epilepsy onsets and helps preventing them by means of meaningful, personalized feedback. It is not hazardous, as it does not introduce risks associated with medication interaction. It is able to identify the individual migraine triggers and/or indivival epilepsy triggers for each patient, i.e. a personalized approach. It prevents migraines and/or epilepsy by tackling the identified triggers (e.g. if trigger is stress and system predicts patient runs high risk to develop migraine and/or epilepsy where trigger is stress, then the system supports the patient to de-stress/relax.). It is a personalized system.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication Systems.

Any reference signs in the claims should not be construed as limiting the scope.

1. System for detecting and preventing a migraine episode in a user, comprising:
a brain activity sensor for detecting a brain activity signal;
a migraine trigger detection unit for detecting potential migraine triggers, said potential migraine triggers being circumstances that may potentially cause a migraine attack in the user;
a processing unit configured to:
(i) process the brain activity signal to identify one or more signal characteristics in the brain activity signal that are indicative of an upcoming migraine attack in the user;
(ii) identify one or more correlations between the one or more identified signal characteristics in the brain activity signal and the detected potential migraine triggers;
(iii) determine, based on the identified one or more correlations, one or more personal migraine triggers that are likely to cause a migraine attack in the user;
an output unit for providing the user with a personalized feedback informing the user about the determined one or more personal migraine triggers and/or how the user may prevent the determined one or more personal migraine triggers.

2. System of claim 1, wherein the brain activity sensor includes an EEG sensor and the brain activity signal includes an EEG signal.

3. System of claim 2, wherein the processing unit is configured to process the EEG signal to identify the one or more signal characteristics in the EEG signal by analysing one or more of (i) the delta and theta frequency bands of the EEG signal, (ii) the alpha waves of the EEG signal, and (iii) the contingent negative variation of the EEG signal.

4. System of claim 1, wherein the migraine trigger detection unit comprises one or more detection sensors for detecting one or more detection signals, and wherein the migraine trigger detection unit is configured to detect the potential migraine triggers by means of a signal analysis of the one or more detection signals.

5. System of claim 4, wherein the one or more detection sensors comprises a vital sign sensor for detecting a vital sign signal of the user, the vital sign sensor including one or more of: (i) a heart rate sensor, (ii) a blood pressure sensor, (iii) a galvanic skin response sensor, and (iv) a photoplethysmographic sensor.

6. System of claim 4, wherein the one or more detection sensors comprises an environmental sensor for detecting an environmental influence in the environment of the user, the environmental sensor including one or more of: (i) a microphone, (ii) a light sensor, (iii) an olfactory sensor, (iv) a tactile sensor, (v) a temperature sensor, and (vi) a humidity sensor.

7. System of claim 4, wherein the one or more detection sensors include a sleep quality sensor for detecting a signal indicative of a sleep quality of the user.

8. System of claim 4, wherein the migraine trigger detection unit comprises an input unit that enables the user to manually enter personalized information on at least one of (i) a sleep quality of the user, (ii) dietary habits of the user, and (iii) other incidents including potential migraine triggers, and wherein the migraine trigger detection unit is configured to detect the potential migraine triggers based on the manually entered personalized information.

9. System of claim 1, further comprising a bracelet, chest strap and/or watch which comprises the migraine trigger detection unit.

10. System of claim 1, further comprising a headset or headband comprising the brain activity sensor.

11. System of claim 1, wherein the output unit comprises one or more of (i) a display, (ii) a loudspeaker, and (iii) a tactile actuator.

12. System of claim 1, further comprising a storage unit for storing the personalized feedback, and a feedback evaluation unit for evaluating an influence of personalized feedback on the user by comparing the personalized feedback stored in the storage unit with the brain activity signal and/or detected potential migraine triggers.

13. System of claim 12, wherein the feedback evaluation unit further comprises an internet module for sharing the stored personalized feedback.

14. Method for detecting and preventing a migraine episode in a user, comprising the steps of:
detecting a brain activity signal of a user;
processing the brain activity signal to identify one or more signal characteristics in the brain activity signal that are indicative of an upcoming migraine attack in the user;
detecting potential migraine triggers, said potential migraine triggers being circumstances that may potentially cause a migraine attack in the user;
identifying one or more correlations between the one or more identified signal characteristics in the brain activity signal and the detected potential migraine triggers;
determining, based on the identified one or more correlations, one or more personal migraine triggers that are likely to cause a migraine attack in the user;
providing the user with a personalized feedback informing the user about the determined one or more personal migraine triggers and/or how the user may prevent the migraine onset by countering the determined one or more personal migraine triggers.

15. Computer program comprising program code means for causing a computer to carry out the steps of the method as claimed in claim 14 when said computer program is carried out on a computer.
